(19) United States
(12) Patent Application Publication
(10) Pub. No.: US 2012/0215045 A1
(43) Pub. Date: Aug. 23, 2012

(54) Staged Injection of Oxygen for Oxidative Coupling or Dehydrogenation Reactions

(75) Inventor: James R. Butler, League City, TX (US)

(73) Assignee: Fina Technology, Inc., Houston, TX (US)

(21) Appl. No.: 13/031,865

(22) Filed: Feb. 22, 2011

(51) Int. Cl. C07C 2/76; B01J 19/00

(52) U.S. Cl. 585/422; 585/721; 422/129

(57) ABSTRACT

Methods and apparatus of staged injection of an oxidant into a feedstream within a reactor are disclosed. The staged injection of the oxidant can better disperse the catalytic reactions throughout the catalyst bed. The staged injection of the oxidant can lower the content of carbon oxides in the reaction product stream, which can reduce energy release from the reactor.

### FIELD

The present invention relates to oxidative coupling and dehydrogenation of hydrocarbons. Specifically, the invention relates to the oxidative coupling of toluene and methane and/or the dehydrogenation of hydrocarbons such as ethylbenzene to styrene.

### BACKGROUND

Styrene, also known as vinyl benzene, is an aromatic compound that is produced in industrial quantities. Polystyrene is a well-known plastic made from the polymerization of the monomer styrene. Polystyrene is one of the largest volume thermoplastic resins in commercial production today. Polystyrene is a durable and inexpensive polymer that is frequently encountered in daily life. Some of the varied applications of polystyrene include insulation, foam cups, disposable cutlery, food packaging, office supplies, CD/DVD cases, housewares, appliance linings, cosmetics packaging, toys, computer housings, bottles, tubing, and dunnage used to protect and secure cargo during transportation.

Styrene is commonly produced by making ethylbenzene, which is then dehydrogenated to produce styrene. Ethylbenzene is typically formed by one or more aromatic conversion processes involving ethylene and the alkylation of benzene. These processes typically involve catalysts, multiple reactions, and substantial energy input. The benzene used to produce ethylbenzene is often produced by the hydrodealkylation of toluene, which requires heating the toluene with excess hydrogen in the presence of a catalyst. This reaction requires more energy input and produces methane as a byproduct.

Toluene is a common byproduct from the production of gasoline or other high value hydrocarbons. As mentioned above, toluene may be used to produce benzene, which can be used to produce ethylbenzene, which can be used to produce styrene. Thus, it would be desirable to economically produce styrene or ethylbenzene directly from toluene. Processes have been developed for oxidative methylation of toluene (OMT), but these processes frequently suffer from poor selectivity for styrene. These known processes also can suffer from large heat losses and can undesirably overoxidize methane to produce carbon oxides.

It is desirable to have a process of producing styrene and ethylbenzene from an oxidative methylation of toluene with less oxidation of methane to carbon oxides. It is also desirable if the process released less heat and is more selective for styrene than current known processes. It is also desirable to have a process of oxidative dehydrogenation, such as dehydrogenation of ethylbenzene to produce styrene, having increased efficiency and conversions. It is also desirable if the process required less steam and could be operated at reduced temperatures than current known processes.

### SUMMARY OF THE INVENTION

The present invention generally relates to methods and processes of staged injection of an oxidant into a feedstream within a reactor to improve selectivity for desired hydrocarbons, and lower the carbon oxides of the effluent, which reduces energy release.

An embodiment, either by itself or in combination with any other aspect of the invention, is a method of oxidatively coupling hydrocarbons that includes providing a reactor with a hydrocarbon inlet, a product stream outlet, a plurality of oxidant injection sites, and an oxidative catalyst. A hydrocarbon feedstream of toluene and methane is fed to the reactor through the hydrocarbon inlet, wherein the toluene and methane oxidatively couple in the presence of the oxidative catalyst and injected oxidant according to a set of reaction conditions. Following the reaction, a product stream containing ethylbenzene and styrene is recovered through the product stream outlet. The plurality of oxidant injection sites may be located on an oxidant supply line that is substantially parallel to the hydrocarbon feedstream and/or substantially concentric with the reactor. Furthermore, the plurality of injection sites may be helically or axially spaced along the oxidant supply line. The oxidant injected into the reactor includes oxygen, but may also include other gases. The oxidant may be injected at high linear velocities into the hydrocarbon feedstream. Moreover, an oxidative catalyst can be placed outside the oxidant supply line. The oxidative catalyst may be in the form of a membrane or coating. The oxidative catalyst may also include pellets, powders, or a combination thereof.

Another embodiment, either by itself or in combination with any other aspect of the invention, is a method of oxidative dehydrogenation of hydrocarbons that includes providing a reactor with a steam inlet, a product stream outlet, a plurality of ethylbenzene injection sites, and a dehydrogenation catalyst. Steam is fed to the reactor through the steam inlet wherein ethylbenzene is injected. The ethylbenzene is oxidatively dehydrogenated in the presence of the steam and the dehydrogenation catalyst according to a set of reaction conditions. Following the reaction, a product stream containing styrene is recovered through the product stream outlet. The plurality of oxygen injection sites may be located on a reactant supply line that is substantially parallel to the steam and/or substantially concentric with the reactor. Furthermore, the plurality of injection sites may be helically or axially spaced along the oxidant supply line. The oxygen may be injected at a high linear velocity. The dehydrogenation catalyst is placed outside the oxidant supply line and may be selected from the group consisting of oxygen, air, carbon dioxide, nitrous oxide, nitrobenzene, and combinations thereof. The dehydrogenation catalyst may be in the form of a membrane or coating. It may also include pellets, powders, or combinations thereof.

Another embodiment, either by itself or in combination with any other aspect of the invention, is a reactor for the oxidative coupling or oxidative dehydrogenation of hydrocarbons. The reactor includes an inlet for feeding at least one hydrocarbon feedstream, an oxidative coupling catalyst, an outlet for recovering a product stream from the reactor, and an oxidant supply line with a plurality of oxidant injection sites between the hydrocarbon feedstream inlet and the product stream outlet. The oxidant supply line may be substantially concentric with the reactor and/or substantially parallel to the hydrocarbon feedstream. The plurality of oxidant injection sites can be axially spaced along the oxidant supply line. The oxidative catalyst may be outside the oxidant supply line. The oxidative catalyst may be in the form of a membrane or coating. The oxidative catalyst may also include pellets, powders, or a combination thereof.

Other possible embodiments include two or more of the above aspects of the invention. In an embodiment the method includes all of the above aspects and the various procedures can be carried out in any order.

### BRIEF DESCRIPTION OF DRAWINGS

FIG. 1 illustrates a flow chart for oxidative coupling of hydrocarbons.

FIG. 2 illustrates a flow chart for oxidative dehydrogenation of hydrocarbons.

FIG. 3 illustrates a reactor having an oxidant supply line substantially parallel with the feedstream flow and concentric with the reactor.

FIG. 4 illustrates an oxidant supply line neither substantially parallel with the reaction flow nor concentric with the reactor.

FIG. 5 illustrates an oxidant supply line where the oxidant injection sites are axially spaced in opposing pairs along the reactant supply line.

FIG. 6 illustrates an oxidant supply line where the oxidant injection sites are spirally spaced along the reactant supply line.

### DETAILED DESCRIPTION

An embodiment of the present invention generally includes staging a plurality of high velocity oxidant injection sites in a reactor in which oxidative coupling of hydrocarbons is occurring. Toluene has been used to produce styrene by reactions with either methanol or methane/oxygen as the co-feed. The latter process is known as oxidative methylation of toluene (OMT).

Theoretically, methanol (CH₃OH) and toluene (C₆H₅CH₃) can be reacted together to form styrene (C₆H₅CH═CH₂), water and hydrogen gas, as shown below:

CH₃OH+C₆H₅CH₃→C₆H₅CH═CH₂+H₂O+H₂

In practice, however, the methanol (CH₃OH) often dehydrogenates into formaldehyde (CH₂O) and hydrogen gas (H₂). Often the toluene conversion is low or the selectivity to products of the methanol is too low to make the process economical. Conversion of methanol to carbon oxides (CO_{X}) or methane can result in an undesirable byproduct stream that is not easily recovered.

Theoretically, methane (CH₄), oxygen (O₂) and toluene (C₆H₅CH₃) may also be reacted to form styrene (C₆H₅CH═CH₂) and water as shown below:

CH₄+C₆H₅CH₃+O₂→C₆H₅CH═CH₂+2H₂O

However, a side reaction of methane with oxygen frequently creates additional heat and carbon oxides. An embodiment of the present invention lessens these effects by injecting oxygen at high linear velocities from a plurality of sites.

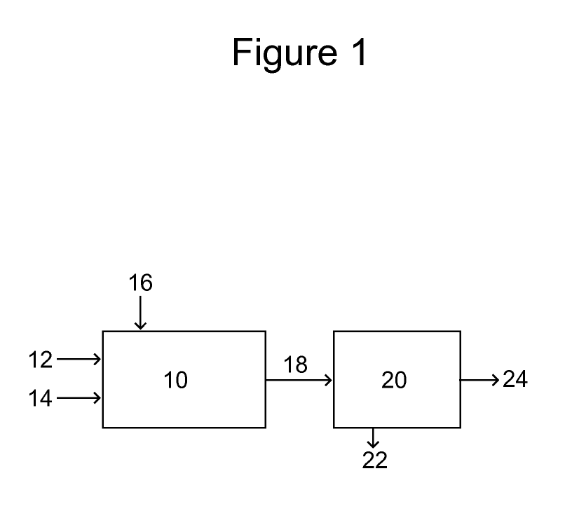

FIG. 1 depicts a simplified flow chart of an oxidative coupling process that may be used to produce styrene from toluene and methane. In an embodiment, a reactor 10 receives a toluene containing stream 12, a methane containing stream 14, and an oxidant stream 16, any of which may be received directly from another proximate process, storage, the ambient environment, or any combination thereof. Furthermore, the toluene stream 12 and methane stream 14 may enter the reactor 10 separately or combined. The toluene and methane are oxidatively coupled in the reactor 10 in the presence of the oxidant stream and an oxidative catalyst (not shown) within the reactor 10. The product stream 18 may then be sent to an optional separation unit 20 where any unwanted byproducts 22 may be separated from the desired products 24, such as styrene. Any unreacted toluene, methane, and oxidant may be separated to be recycled back to the reactor 10. Furthermore, any byproducts 22 such as water, carbon oxides, and hydrogen may also be separated at this point. The oxidant stream 16 can be distributed by use of a plurality of oxidant injection sites (not shown) located within the reactor 10 for contact with the toluene stream 12 and methane stream 14.

The operating conditions of the reactors and separators can be system specific and vary depending on the feedstream composition and the compositions of the product streams. The reactor 10 for the reaction of toluene and methane in the presence of an oxidant may operate at elevated temperatures and pressures, and may contain a basic or neutral catalyst system. In a non-limiting example the temperature can range from 250 to 1000° C., optionally from 400 to 900° C., optionally from 500 to 700° C. The pressure can range from 0.1 atm to 100 atm, optionally from 1.0 atm to 80 atm, optionally from 1.0 atm to 50 atm. The flowrate can range from 1 to 70 LHSV based on toluene flow, optionally from 1 to 50 LHSV, optionally from 2 to 50 LHSV.

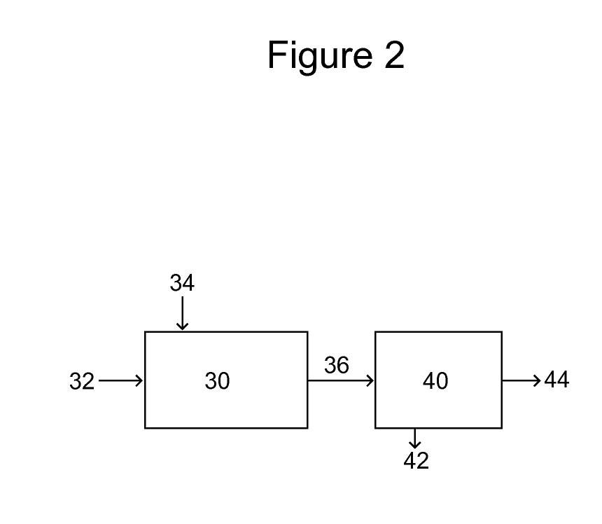

FIG. 2 depicts a simplified flow chart of an oxidative dehydrogenation process that may be used to process many hydrocarbons, including but not limited to producing styrene from ethylbenzene. In an embodiment, a reactor 30 receives an ethylbenzene stream 32 and an oxidative stream 34, either of which may be received directly from storage, another proximate process, or any combination thereof. The oxidative steam 34 oxidatively dehydrogenates the ethylbenzene 32 in the presence of an oxidative catalyst (not shown) within the reactor 30 and under reaction conditions. The product stream 36 may then be sent to an optional separation unit 40 where any unwanted byproducts 42 may be separated from the desired products 44. Styrene is the desired product in this embodiment. Any unreacted ethylbenzene and steam may be separated to be recycled back to the reactor 30 or another dehydrogenation reactor. The oxidative stream 34 can be distributed by use of a plurality of oxidant injection sites (not shown) located within the reactor 30 for contact with the ethylbenzene stream 32.

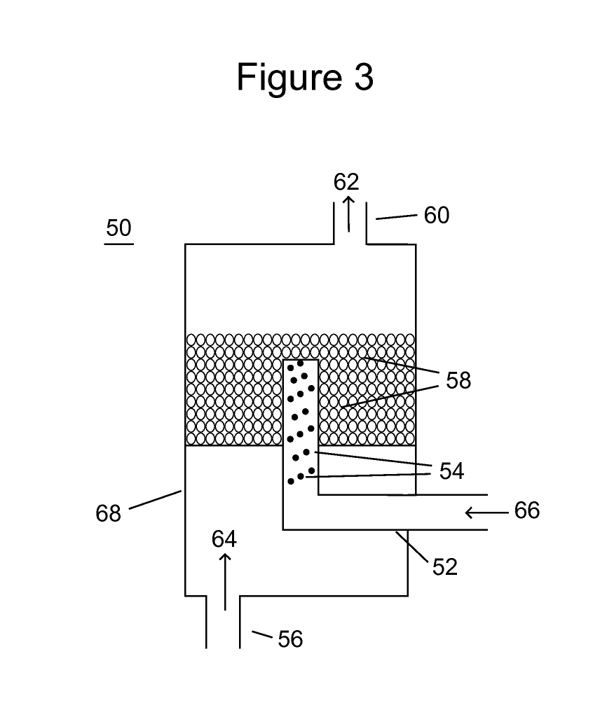

FIG. 3 illustrates an embodiment of the invention wherein a reactor 50 may be used for the oxidative coupling of hydrocarbons. The reactor 50 has an oxidant supply line 52 with a plurality of oxidant injection sites 54. The reactor 50 also has an inlet 56 for the hydrocarbons, an oxidative catalyst 58, and an outlet 60 for the product stream 62. A hydrocarbon feedstream 64 passes through the hydrocarbon inlet 56 into the reactor 50 contained within a reactor wall 68. In this embodiment as a non-limiting example, the hydrocarbon feedstream 64 contains toluene and methane in either a substantially mixed or unmixed state. In an embodiment the toluene and methane are unmixed and can be added to the reactor 50 in separate inlet streams (not shown). In an embodiment, the feedstream 64 is substantially mixed before entering the reactor 50. In an alternate embodiment (not shown) the toluene and methane are added in separate inlet streams to the reactor. The oxidant supply line 52 is located within the reactor 50 wherein the hydrocarbon feedstream 64 oxidizes in the presence of the catalyst 58 to form the product stream 62. The reactant supply line 52 may be substantially concentric with the reactor 50. The oxidant injection sites 54 can be partially or totally located within the oxidative catalyst 58. In the embodiment shown in FIG. 3 a portion of the oxidant injection sites 54 are located adjacent to the oxidative catalyst 58 while a portion of the oxidant injection sites 54 are located not adjacent to the oxidative catalyst 58 so as to contact the hydrocarbon feedstream 64 prior to the hydrocarbon feedstream 64 contacting the oxidative catalyst 58.

The reactor 50 may be any type of reactor known in the art, including but not limited to a fixed bed, plug flow reactor, fluidized bed reactor, or a stirred-tank reactor. The toluene and methane may be supplied in various ratios of from 1:1 methane:toluene to 50:1 methane:toluene, optionally from 1:1 to 30:1 methane:toluene, optionally from 1:1 to 15:1 methane:toluene, optionally from 1:1 to 10:1 methane:toluene.

The oxidative steam 66, which includes oxygen, is added to the reactor 50 through the oxidant supply line 52 in amounts that can facilitate the conversion of toluene and methane to ethylbenzene and styrene. The oxygen can be injected with a high linear velocity into the hydrocarbon feedstream 64 at a plurality of reactant injection sites 54 along the reactant supply line 52 to supply adequate mixing of the oxygen with the hydrocarbon feedstream 64 and the oxidative catalyst 58. The velocity of the oxidant is desirably high enough to enable the oxygen to contact substantially all of the hydrocarbon feedstream 64 and not allow a significant amount of hydrocarbon feedstream 64 to pass through the reactor 50 and the oxidative catalyst 58 without contact with oxygen. In an embodiment the staged injection of the oxidant can lower carbon oxides of the resulting product stream 62, which can reduce energy release from the reactor 50.

As used herein the term “high linear velocity” as referring to the oxidant injection means at a velocity that is able to substantially distribute the oxygen throughout the entire hydrocarbon feedstream. The high linear velocity is desirable to minimize the amount of hydrocarbon feedstream that may not come in contact with the oxidant. The velocity needed to substantially distribute the oxygen throughout the entire hydrocarbon feedstream will be dependent on factors such as the reactor dimensions and the flowrates of the feedstream and oxidant streams.

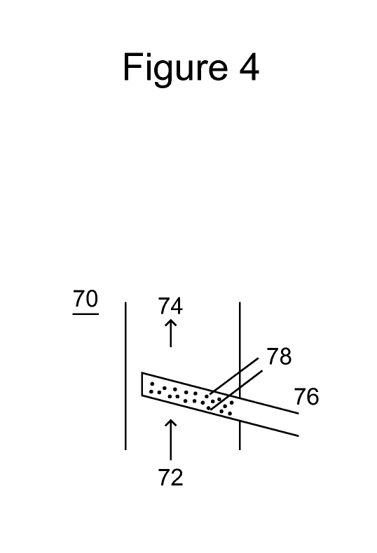

In an alternative embodiment, the oxidant supply line is located within the reactor, but is not substantially concentric with the reactor. FIG. 4 illustrates in a non-limiting example that the oxidant supply line 76 may be neither substantially parallel with the hydrocarbon feedstream flow 72 in a reactor 70 nor substantially concentric with the reactor 70. The plurality of oxidant injection sites may be spaced along the oxidant supply line to effectively disperse the oxidant radially throughout the hydrocarbon feedstream. In yet another alternative embodiment, the oxidant supply line may inject the oxidant into the hydrocarbon feedstream opposite the direction of the hydrocarbon stream flow.

FIGS. 3, 4, 5 and 6 illustrate non-limiting examples of injection site configurations along the oxidant supply line. FIG. 3 illustrates the plurality of injection sites 54 spaced along the oxidant supply line 52 in a helical manner. The injection sites 54 can be spaced along the oxidant supply line 52 in a substantially equidistant manner, or alternately in a non-equidistant manner, or various combinations thereof.

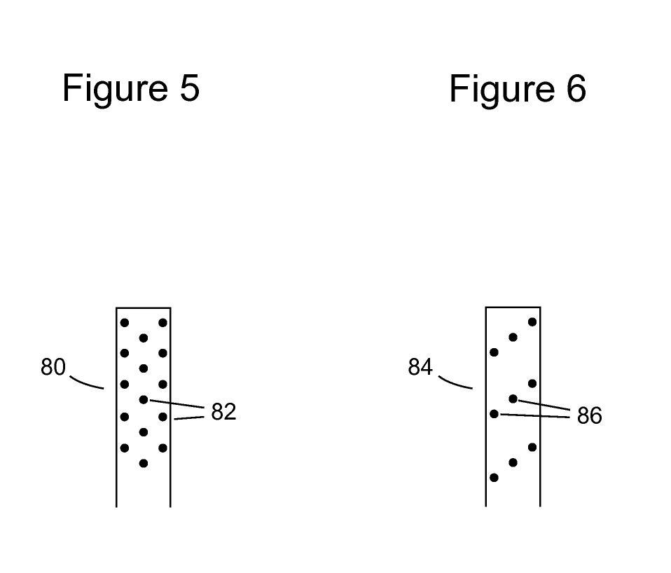

FIG. 4 illustrates a portion of a reactor 70 having a hydrocarbon feedstream 72, a product stream 74, and an oxidant supply line 76 having a plurality of injection sites 78 randomly spaced. FIG. 5 illustrates an oxidant supply line 80 with a plurality of injection sites 82 equidistantly spaced along the oxidant supply line 80 in an axial manner. FIG. 6 illustrates an oxidant supply line 84 with a plurality of injection sites 86 equidistantly spaced along the oxidant supply line 84 in a spiral manner.

Injection of the oxidant at a plurality of injection sites gives greater oxidant dispersion throughout the hydrocarbon feedstream. Furthermore, the plurality of injection sites may better disperse the catalytic reactions throughout the reactor and throughout the catalyst bed. In an embodiment the plurality of injection sites can better disperse the catalytic reactions throughout the reactor and the catalyst bed and can allow for more efficient heat removal.

In a non-limiting embodiment, either by itself or in combination with any other aspect of the invention, the oxygen content can range from 1% to 50% by volume relative to the methane content, optionally from 5% to 40%, optionally from 5% to 25%. The high linear velocity of the oxygen causes the oxidative methylation of toluene to occur rapidly in the presence of the catalyst. The high linear velocity may also minimize the side reaction with methane and decrease the heat and carbon oxides formed. Furthermore, due to the high linear velocity of the oxygen, the reaction is distributed over more of the catalyst and large particle catalysts may be used.

The oxygen may still react with a portion of the methane in an exothermic reaction. The heat generated by this reaction may be dissipated in many ways not shown, such as for example utilizing an external cooling jacket, internal cooling coils, or heat exchange. The heat removal can be controlled in such a manner as to maintain the reaction within a desired temperature range to facilitate the conversion of toluene and methane to ethylbenzene and/or styrene. In an embodiment, the desirable temperature range is from 200° C. to 1000° C., optionally from 250° C. to 800° C., optionally from 500° C. to 700° C. The pressure can range in a non-limiting example from 0.1 atm to 100 atm, optionally from 1 atm to 100 atm, optionally from 1 atm to 70 atm. Moreover, the heat generated by the exothermic reaction can be removed and recovered to be utilized within the methylation process or another process.

In a non-limiting embodiment, either by itself or in combination with any other aspect of the invention, the oxidative catalyst can be located outside the oxidant supply line inside the reactor and between the reactor inlet and outlet. The catalyst may fill all or part of the annulus between the oxidant supply line and the reactor wall in the form of pellets, powders, or combinations thereof. Furthermore, the catalyst may form a coating or membrane on the oxidant supply line, the reactor wall, the wetted parts of the reactor, or any combination thereof. The oxidant supply line may inject oxygen into the hydrocarbon feedstream before the stream contacts the catalyst, where the hydrocarbon feedstream is passing through the catalyst, or at both locations along the oxidant supply line.

In a non-limiting embodiment, either by itself or in combination with any other aspect of the invention, the reactor may include one or more of single or multistage catalyst beds containing the catalyst. Furthermore, more than one type of catalyst may be used. In an embodiment, the catalyst can include any catalyst capable of catalyzing oxidative coupling reactions of hydrocarbons, such as coupling toluene and methane to form ethylbenzene and/or styrene. Such a catalyst may include one or more metal oxides. The oxidative catalyst may contain different combinations of alkali, alkaline earth, rare earth, and/or transition metal oxides. In other non-limiting examples, the catalyst can include a modified basic zeolite, a base zeolite, or zeolites with or without metal oxides.

The oxidative catalyst may be any catalyst capable of catalyzing oxidative coupling reactions of hydrocarbons. In a non-limiting embodiment, either by itself or in combination with any other aspect of the invention, the oxidative catalyst includes: (A) at least one element selected from the group consisting of the Lanthanoid group, Mg, Ca, and the elements of Group 4 of the periodic table (Ti, Zr, and Hf), the elements from (A) ranging from 40 to 90 wt % of the catalyst; (B) at least one element selected from the group consisting of the Group 1 elements of Li, Na, K, Rb, Cs, and the elements of Group 3 (including La and Ac) and Groups 5-15 of the periodic table, the elements from (B) ranging from 0.01 to 40 wt % of the catalyst; (C) at least one element selected from the group consisting of the Group 1 elements of Li, Na, K, Rb, Cs, and the elements Ca, Sr, and Ba, the elements from (C) ranging from 0.01 to 40 wt % of the catalyst; and (D) oxygen ranging from 10 to 45 wt % of the catalyst; wherein if an element from Group 1 of the periodic table is used in (B), it cannot be used in (C); wherein the catalyst is calcined after the elements are combined. The elements from (A) can range from 40 to 90 wt % of the catalyst, optionally from 40 to 75 wt % of the catalyst, optionally from 40 to 50 wt % of the catalyst. The elements from (B) can range from 0.01 to 40 wt % of the catalyst, optionally from 0.1 to 30 wt % of the catalyst, optionally from 1.0 to 20 wt % of the catalyst. The elements from (C) can range from 0.01 to 40 wt % of the catalyst, optionally from 0.1 to 30 wt % of the catalyst, optionally from 1.0 to 20 wt % of the catalyst. The oxygen from (D) can range from 10 to 45 wt % of the catalyst, optionally from 15 to 40 wt % of the catalyst, optionally from 20 to 30 wt % of the catalyst.

Irrespective of how the components are combined and irrespective of the source of the components, the dried composition is generally calcined in the presence of a free oxygen-containing gas, usually at temperatures between about 300° C. and about 900° C. for from 1 to 24 hours. The calcination can be in an oxygen-containing atmosphere, or alternately in a reducing or inert atmosphere. Upon calcination these elements can be altered, such as through oxidation which would increase the relative content of oxygen within the final catalyst structure. The combination of the catalyst of the present invention combined with additional elements such as a binder, extrusion aid, structured material, or other additives, and their respective calcination products, are included within the scope of the invention.

Styrene is also formed industrially through the dehydrogenation of ethylbenzene. In the dehydrogenation process, ethylbenzene may be mixed with steam in the presence of a metal oxide catalyst under dehydrogenation conditions to form styrene. The metal oxide catalyst is frequently an iron oxide. The catalyst serves to strip hydrogen from the ethyl group on the benzene ring. This forms a styrene molecule with its characteristic double carbon bond. Other side reactions may occur due to impurities. The main reaction takes ethylbenzene (C₆H₅CH₂CH₃) to form styrene (C₆H₅CH═CH₂) and hydrogen (H₂). The hydrogen can be separated and can be used for any suitable purpose, such as for heating steam or other processes. Dehydrogenation reactors are frequently used in series to obtain the desired styrene concentration of the product stream.

The catalyst of the present embodiment may include an iron compound, an alkali metal compound, and optionally a cerium compound.

In an alternate embodiment aspects of the present invention can be applied to oxidative dehydrogenation reactions. Referring to FIG. 3, a reactor 50 has an oxidant supply line 52 with a plurality of oxidant injection sites 54. The reactor 50 also has an inlet 56 for the hydrocarbons, an oxidative catalyst 58, and an outlet 60 for the product stream 62. A hydrocarbon feedstream 64 passes through the hydrocarbon inlet 56 into the reactor 50. In this embodiment as a non-limiting example, the hydrocarbon feedstream 64 contains ethylbenzene. The oxidant supply line 52 is located within the reactor 50 wherein a portion of the hydrocarbon feedstream 64 undergoes a dehydrogenation reaction in the presence of the catalyst 58 to form the product stream 62 that contains styrene. The dehydrogenation catalyst 58 is located outside the oxidant supply line 52 inside the reactor 50. This catalyst 58 may fill all or part of the annulus between the reactant supply line 52 and the reactor wall 68 in the form of pellets, powders, or combinations thereof. Moreover, the dehydrogenation catalyst 58 may also surround the wetted portion of the oxidant supply line 52 within the reactor in any other manner. Furthermore, the catalyst 58 may form a coating or membrane on the oxidant supply line 52, the reactor wall 68, the wetted parts of the reactor, or any combination thereof.

The reactor 50 may include one or more of single or multistage catalyst beds containing the catalyst 58. Furthermore, more than one type of catalyst 58 may be used. In an embodiment, the catalyst 58 can include any catalyst that aids in dehydrogenating hydrocarbons, such as dehydrogenating ethylbenzene to form styrene. The dehydrogenation catalyst may be of any suitable type, typically constituting an iron oxide-based catalyst comprising iron oxide or a mixture of iron oxide with chromium oxide and sodium oxide, such as disclosed in U.S. Pat. No. 4,549,032 to Moeller, incorporated herein by reference. The dehydrogenation catalyst may incorporate iron oxide along with secondary components such as chrome oxide as well as other inorganic materials and can be formulated with a binder into desirable sizes, such as for example particle sizes of about ⅛-inch. One non-limiting example of a catalyst for use in carrying out the present invention is an iron oxide catalyst promoted with potassium carbonate plus trace metals for selectivity enhancement available from CRI Catalyst Company under the designation “Flexicat Yellow.”

The product stream 62 may include styrene, ethylbenzene, toluene, and benzene, among other byproducts such as hydrogen and steam. Styrene may be separated out for manufacture of polystyrene, whereas the other components of the product stream may be further separated in a separator unit to be recycled in subsequent processes for later use or disposal.

The operating conditions of the reactors and separators can be system specific and vary depending on the feedstream composition and the compositions of the product streams. The dehydrogenation reaction can take place according to a set of reaction conditions, which include feedstock specifications, temperature, pressure, and space velocity. Generally these conditions are known in the art, but the following are some non-limiting conditions. Dehydrogenation reactions are generally endothermic, and the temperature in the reactor can be from 300° C. to 1000° C., optionally from 400° C. to 900° C., optionally from 500° C. to 700° C. The required heat is typically provided by steam, but the reactants may otherwise be preheated before entering the reactor. The pressure can be above atmospheric or sub-atmospheric, such as from 0.1 atm to 10 atm, optionally from 0.1 atm to 5.0 atm, optionally from 0.5 atm to 1.5 atm.

In an alternative embodiment, the oxidant supply line 52 is located within the reactor 50, but is not substantially concentric with the reactor 50. Furthermore, the reactant supply line may not be substantially parallel to the flow of the steam through the reactor. In another embodiment, the plurality of reactant injection sites 54 may be spaced along the oxidant supply line 52 to effectively disperse the oxidant radially throughout the hydrocarbon feedstream 64. In yet another alternative embodiment, the oxidant supply line 52 may inject the oxidant into the hydrocarbon feedstream 64 opposite the direction of the hydrocarbon feedstream flow.

Various terms are used herein, to the extent a term used is not defined herein, it should be given the broadest definition persons in the pertinent art have given that term as reflected in printed publications and issued patents. Various ranges are further recited herein. It should be recognized that unless stated otherwise, it is intended that the endpoints are to be interchangeable. Further, any point within that range is contemplated as being disclosed herein

Use of broader terms such as comprises, includes, having, etc. should be understood to provide support for narrower terms such as consisting of, consisting essentially of, comprised substantially of, etc.

Use of the term “optionally” with respect to any element of a claim is intended to mean that the subject element is required, or alternatively, is not required. Both alternatives are intended to be within the scope of the claim.

The term “deactivated catalyst” refers to a catalyst that has lost enough catalyst activity to no longer be efficient in a specified process. Such efficiency is determined by individual process parameters. A deactivated catalyst generally requires process shut down in order for a regeneration procedure to be carried out.

While illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Depending on the context, all references herein to the “invention” may in some cases refer to certain specific embodiments only. In other cases it may refer to subject matter recited in one or more, but not necessarily all, of the claims. While the foregoing is directed to embodiments of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the aspects and embodiments disclosed herein are usable and combinable with every other embodiment and/or aspect disclosed herein, and consequently, this disclosure is enabling for any and all combinations of the embodiments and/or aspects disclosed herein. Other and further embodiments, versions and examples of the invention may be devised without departing from the basic scope thereof and the scope thereof is determined by the claims that follow.

1. A method of producing a hydrocarbon product comprising:
providing a reactor comprising a reactant inlet and a product outlet;
providing a catalyst within the reactor;
feeding a hydrocarbon feed to the reactor through the reactant inlet;
providing an oxidant supply line comprising a plurality of oxidant injection sites;
feeding an oxidant to the reactor through the plurality of oxidant injection sites;
carrying out an oxidative reaction of the hydrocarbon feed over the catalyst according to a set of reaction conditions; and
recovering a hydrocarbon product from the reactor through the product outlet;
wherein the plurality of oxidant injection sites are located between the reactant inlet and the product outlet.

2. The method of claim 1, wherein the oxidant supply line is substantially concentric with the reactor.

3. The method of claim 1, wherein the plurality of oxidant injection sites are helically spaced along the oxidant supply line.

4. The method of claim 1, wherein the plurality of oxidant injection sites are axially spaced along the oxidant supply line.

5. The method of claim 1, wherein the oxidant supply line is substantially parallel to hydrocarbon flow through the reactor.

6. The method of claim 1, wherein the oxidant is supplied at high linear velocity into the hydrocarbon flow through the reactor.

7. The method of claim 1, wherein the catalyst is placed outside the oxidant supply line.

8. The method of claim 1, wherein the catalyst is a membrane or coating.

9. The method of claim 1, wherein the catalyst comprises pellets, powders, or a combination thereof.

10. The method of claim 1, wherein the catalyst is an oxidative catalyst.

11. The method of claim 10, wherein the hydrocarbon feed comprises methane and the hydrocarbon product comprises C₂ hydrocarbons.

12. The method of claim 10, wherein the hydrocarbon feed comprises toluene and the hydrocarbon product comprises ethylbenzene and styrene.

13. The method of claim 1, wherein at least a portion of the oxidant injection sites are located adjacent to the catalyst.

14. The method of claim 1, wherein the catalyst is a dehydrogenation catalyst.

15. The method of claim 14, wherein the hydrocarbon feed further comprises steam.

16. The method of claim 14, wherein the hydrocarbon feed comprises ethylbenzene and the hydrocarbon product comprises styrene.

17. The method of claim 1, wherein feeding an oxidant to the reactor through the plurality of oxidant injection sites lowers the content of carbon oxides in the hydrocarbon product, which reduces energy release from the reactor.

18. An apparatus comprising:
a reactor comprising a reactant inlet and a product outlet;
an oxidant supply line comprising a plurality of oxidant injection sites;
wherein the plurality of oxidant injection sites are located within the reactor between the reactant inlet and the product outlet.

19. The apparatus of claim 17, wherein the oxidant supply line is substantially concentric with the reactor.

20. The apparatus of claim 17, wherein the plurality of oxidant injection sites are axially spaced along the oxidant supply line.

21. The apparatus of claim 17, wherein the oxidant supply line is substantially parallel to hydrocarbon flow through the reactor.
